(19) United States
(12) Patent Application Publication
(10) Pub. No.: US 2019/0059908 A1
(43) Pub. Date: Feb. 28, 2019

(54) APPARATUS, SYSTEM AND METHOD FOR TREATING HEMORRRHAGE

(71) Applicant: UTI Limited Partnership, Calgary (CA)

(72) Inventors: Nigel CAULKETT, Calgary (CA); Jessica PANG, Calgary (CA); Soren BOYSEN, Calgary (CA)

(21) Appl. No.: 16/078,567

(22) PCT Filed: Feb. 22, 2017
(86) PCT No.: PCT/CA2017/050222

§ 371 Date: Aug. 21, 2018

Related U.S. Application Data
(60) Provisional application No. 62/298,225, filed on Feb. 22, 2016.

(51) Int. Cl. A61B 17/12

(52) U.S. Cl. CPC A61B 17/12136; A61B 2017/1205; A61B 2017/12004; A61B 17/1204

(57) ABSTRACT

A system for decreasing blood flow that comprises a transesophageal aortic compression balloon, wherein the balloon comprises a body and an expandable section that can cycle between an expanded state and a compressed state. The system further includes a controller for actuating the expandable section between the expanded state and the compressed state according to a predetermined pattern. When the expanded balloon is adjacent a subject's aorta it may at least partially compress a portion of the subject's aorta and decrease blood flow below the point of compression.

### CROSS REFERENCES TO RELATED APPLICATIONS

This application is a national stage application of co-pending International Application No. PCT/CA2017/050222, filed Feb. 22, 2017, which is incorporated herein by reference in its entirety, and additionally claims the benefit of and priority to U. S. Provisional Patent Application No. 62/298,225 filed Feb. 22, 2016, which is incorporated herein by reference in their entirety.

### TECHNICAL FIELD

The present disclosure relates to the field of surgical intervention. In particular, the present disclosure relates to the field of surgical intervention to treat one or more hemorrhage.

### BACKGROUND

Exsanguination due to hemorrhage is a major cause of civilian trauma-death and approximately 50% of battlefield trauma-deaths. Many of these deaths may be prevented with early intervention. In the face of massive ongoing hemorrhage surgical intervention is a typical means used to stabilize the patient. However, pre-operative stabilization is also important to a successful outcome in trauma patients. Pre-operative stabilization for a massive hemorrhage often includes transfusing crystalloid fluids or stored blood products into a subject until definitive surgical repair of the hemorrhage can be performed. These interventions are not without their own risks or drawbacks. For example, transfusing large volumes of crystalloid fluids can result in abdominal compartment syndrome, which is a potentially life threatening condition. Transfusing large volumes of blood products may not always be a viable option because blood products can be difficult to obtain and store and large volume transfusions may also result in coagulopathies that could worsen the subject's prognosis.

For these reasons recent research has focused on interventions that will reduce blood loss, maintain the patient's own blood volume, and reduce the volume of fluids or blood products required for resuscitation. For example, the administration of tranexamic acid, a drug that inhibits fibrinolysis following hemorrhage, has been shown to decrease blood transfusion requirements and the risk of death in people who have significant injury secondary to trauma.

Other interventions have focused on vascular compression within the abdomen to control severe intra-abdominal hemorrhage.

One such intervention utilizes the injection of a polyurethane foam into the abdominal cavity to control abdominal hemorrhage. This intervention is effective but can induce focal bowel injuries, which if severe enough may require a surgical resection that carries its own inherent risks.

Another approach to control hemorrhage is to employ expandable devices. US 20130310872 describes a portable pneumatic abdominal aortic compression system. This system is applied externally to the abdominal wall to provide counter-pressure. The system includes a balloon-based compression device that is wrapped externally around the abdomen and that applies pressure on the abdomen and aorta in an effort to control bleeding. While this compression device may assist in stabilizing a trauma subject for transport, the external placement of the compression device may limit its usefulness for maintaining hemostasis in preparation for and during surgery.

U.S. Pat. No. 5,716,386 A discloses an expandable device that is positioned within the subject's esophagus during cardiopulmonary resuscitation (CPR). The device may be expanded by injecting a fluid into a balloon portion. The expanded balloon at least partially occludes the subject's descending thoracic aorta in an effort to improve blood flow to the heart and lungs during CPR. The device may also cool a subject's blood during CPR to decrease cerebral metabolic rate in an effort to improve the subject's outcome.

Further examples of known expandable devices for treating hemorrhage include WO2008100433A2 and US20050143689. WO2008100433A2 discloses a pelvic balloon tamponade for treating post-partem hemorrhage. US20050143689 discloses an internal compression-tourniquet catheter system. These expandable devices may provide compression and hemorrhage control at a specific site or organ.

### SUMMARY

Embodiments of the present disclosure relate to an apparatus, system and method for treating hemorrhage.

Some embodiments of the apparatus comprise an expandable section that can be cycled between an expanded state and a compressed state. In the expanded state the expandable section can temporarily occlude a portion of a subject's aorta.

Without being bound by any particular theory, it has been observed that maintaining the expandable section in the expanded state for a prolonged period causes a negative effect on carotid blood flow, which is an indication of reduced cerebral blood flow. Accordingly, the expandable section may be cycled between the expanded state and the compressed state.

In some embodiments of the present disclosure, the apparatus may be positioned within the subject's esophagus so that the expandable section is positioned proximal the aortic hiatus of the subject's diaphragm. When the expandable section is cycled to the expanded state at this position the descending aorta may be at least partially occluded and reduce blood loss into the abdominal cavity. This positioning of the expandable section may also reduce blood loss from the distal limbs. The cyclic expanding and compressing of the expandable section may avoid impairing cardiac or cerebral vascular blood flow.

Some embodiments of the present disclosure relate to a system for decreasing blood flow. The system comprises an apparatus that comprises a body and an expandable section capable of cycling between an expanded state and a compressed state. The system further includes a controller for actuating the expandable section between the expanded state and the compressed state according to a predetermined pattern. When the expanded apparatus is adjacent a subject's aorta it may at least partially compress a portion of the subject's aorta and decrease blood flow below the point of compression.

Some embodiments of the present disclosure relate to a method of controlling hemorrhage within a subject. The method comprises the steps of: inserting a transesophageal balloon within the subject's esophagus adjacent the subject's aorta; expanding the transesophageal balloon for establishing a point of compression of the subject's aorta and reducing blood flow beyond the point of compression; and contracting the transesophageal balloon for resuming blood flow beyond the point of compression.

### BRIEF DESCRIPTION OF THE DRAWINGS

These and other features of the present disclosure will become more apparent in the following detailed description in which reference is made to the appended drawings.

FIG. 1 shows a side elevation view of an example embodiment of an apparatus of the present disclosure with one end shown as a partial cut-away to reveal internal structures thereof: A) shows the apparatus in an expanded state; and B) shows the apparatus in a compressed state;

FIG. 2 shows a side elevation view of another embodiment of the apparatus of FIG. 1 with one end partially cut away to show internal structures thereof: A) shows the apparatus in the expanded state; and B) shows the apparatus in the compressed state;

FIG. 3 shows a side elevation view of one embodiment of a system of the present disclosure with one end shown as a partial cut-away to reveal internal structures thereof;

FIG. 4 shows a side elevation view of an embodiment of an apparatus of the present disclosure in relation to subject's anatomical structures when positioned within the subject: A) shows the apparatus in the compressed state; and B) shows the apparatus in the expanded state; and

FIG. 5 is a view taken along line 5-5¹ in FIG. 4 that is a bottom plan view of the subject's diaphragm: A) shows the apparatus in the compressed state, as shown in FIG. 4A; and B) shows the apparatus in the expanded state, as shown in FIG. 4B.

### DETAILED DESCRIPTION

Embodiments of the present disclosure relate to an apparatus, system and method for treating a hemorrhage. One embodiment provides an apparatus that comprises a transesophageal aortic compression-balloon (TAC-B). A TAC-B is a simple and relatively non-invasive device that may decrease hemorrhage in the abdomen or in the distal limbs in a deeply sedated or anesthetized subject. The TAC-B comprises an expandable section that can cyclically actuate between an expanded state and a compressed state. Optionally the expandable section can actuate by cyclic inflation and deflation according to a predetermined pattern. Cyclic inflation and deflation of the TAC-B may reduce blood flow distal to the aortic hiatus of the diaphragm while maintaining venous return. Venous return maintains the critical blood flow needs of the heart, lungs and brain. Intraoperatively, the expandable section can be inflated and maintained in the expanded state for desired periods of time in order to achieve transient hemostasis from an abdominal or distal vessel that is hemorrhaging. Even transient periods of hemostasis may provide a surgeon time and a clear field to locate the ruptured vessel, surgically place shunts, or implement other means to achieve longer lasting hemostasis.

### Definitions

Unless defined otherwise, all technical and scientific terms used herein have the same meaning as commonly understood by one of ordinary skill in the art to which this disclosure belongs.

As used herein, the term “about” refers to an approximately +/−10% variation from a given value. It is to be understood that such a variation is always included in any given value provided herein, whether or not it is specifically referred to.

As used herein, the term “hemostasis” describes when hemorrhaging from a blood vessel that has lost containment properties is stopped, either permanently or temporarily.

As used herein, the term “hemorrhage” refers to loss of blood volume through one or more blood vessels that have lost fluid containment properties due to be being cut, split, severed, ruptured or otherwise damaged so that the total blood volume within the circulatory system decreases over time.

As used herein, the term “subject” refers to a human or veterinary patient.

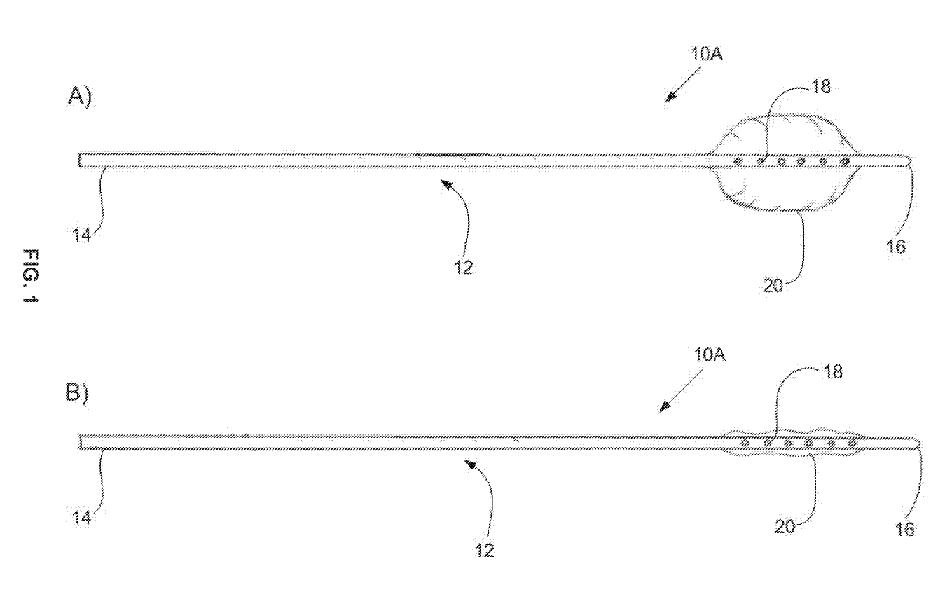

Embodiments of the present disclosure will now be described by reference to FIG. 1 to FIG. 5B, which show representations of an apparatus, system and method for treating hemorrhage according to the present disclosure.

FIG. 1A and FIG. 1B show one embodiment of a TAC-B 10A. The TAC-B 10A comprises a body 12 and an expandable section 20 that is connected to one end of the body 12.

The body 12 has a first end 14 and a second end 16. The body 12 is elongate and tubular with a central bore (not shown) that provides fluid communication between the first and second ends 14, 16. The body 12 may also be referred to as a cannula. In one embodiment, the body 12 may be constructed of semi-rigid material, such as polyvinyl chloride (PVC) or silicon. The body 12 has a total length between the first and second ends 14, 16 that can be in a range from about 25 cm to about 125 cm. The body 12 may have an external diameter that ranges from about 0.25 cm to about 1.75 cm. The body 12 may optionally be marked with line markings of 1 cm or 5 cm increments.

The second end 16 may have a blunt tip to facilitate insertion of the body 12 within and along the esophagus of a subject. The second end 16 may also define one or more apertures 18 that provide fluid communication from the central bore to outside of the body 12. In one embodiment there are a plurality of apertures 18 that are positioned within a distance from the second end 16 that is substantially equivalent to about ⅙ to about ⅓ of the total length of the body 12.

The expandable section 20 is positioned about the body 12 above the apertures 18. The expandable section 20 attached in a fluid tight fashion against the outer surface of the body 12 so that the area between the expandable section 20 and the central bore are in fluid communication. The expandable section 20 is configured to receive and retain fluid that moves through the apertures 18 from the central bore inside the body 12, which causes the expandable section 20 to expand. Fluid can also leave the expandable section 20 by moving through the apertures 18, which will actively or passively compress the expandable section 20. The movement of fluids into the apertures 18 from the expandable section 20 may also be referred to as release of the fluids from the expandable section 20. As will be appreciated by one of skill in the art, the movement of fluid into and out of the expandable section 20 can occur by various means. The fluid may be a gas or liquid. In some embodiments of the present disclosure the fluid is a gas that is pumped into and out of the expandable section 20, as will be described in further detail below.

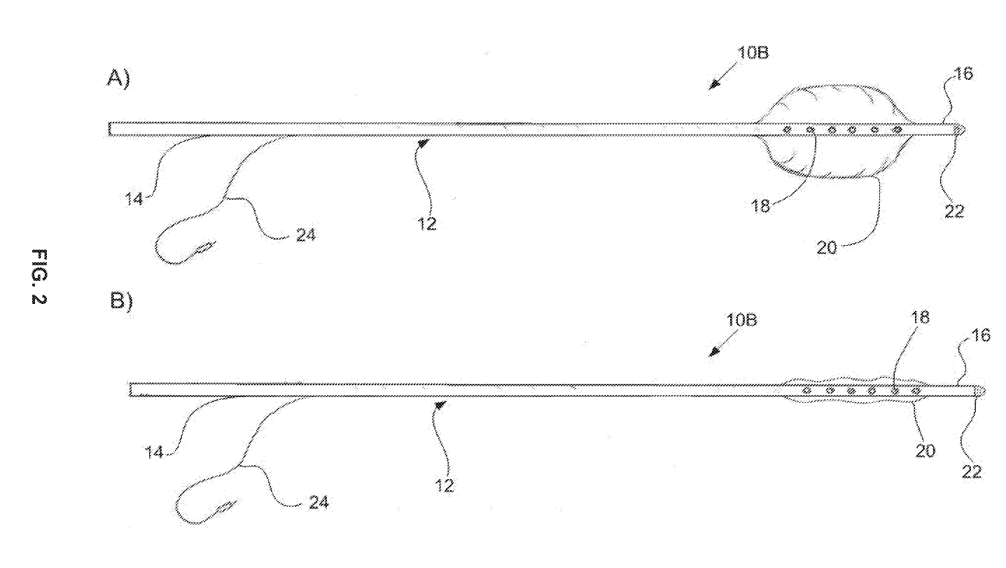

FIG. 2A and FIG. 2B show an alternative embodiment of a TAC-B 10B that has many of the same features as the TAC-B 10A described above and shown in FIG. 1A and FIG. 1B. TACB-10B further comprises a sensor 22 that is positioned at or near the second end 16. The sensor 22 detects positional information of the second end 16 and provides a positional signal to a user to assist in positioning the TACB-10B during use. In one exemplary embodiment, the sensory 22 is a pH sensor that can detect a change in the pH of the environment proximal to the second end 16. For example, the sensor 22 can detect a decrease in pH when the sensor 22 enters the stomach. This drop in pH generates a signal that is directed to the user, for example via a sensor output 24. Sensor output 24 may provide the signal in a format that the user may understand. For example, the sensor output 24 signal output format may be an electronic signal that is converted into a visual, auditory or other format that can be presented to the user.

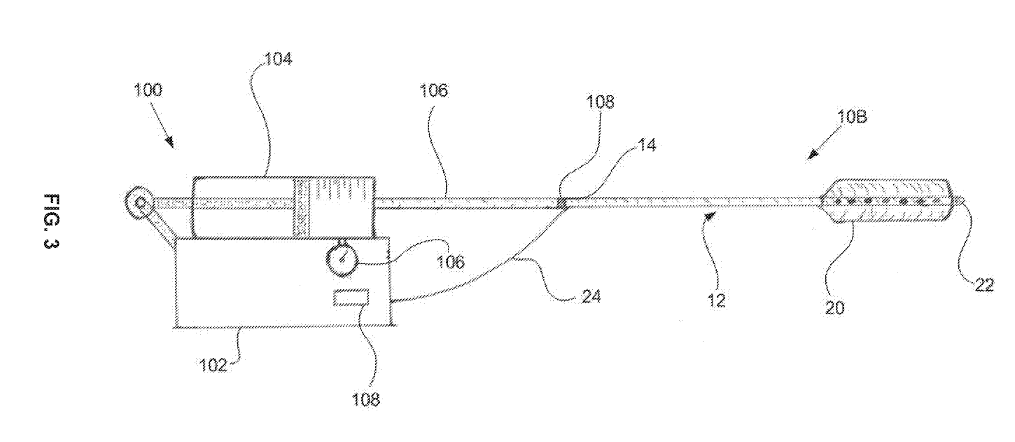

FIG. 3 shows the TAC-B 10B as part of a system 100. It is understood that the TAC-B 10A may also be used as part of the system 100. The system 100 further comprises a controller 102 that controls actuation of the expandable section 20. For example, the controller 102 may provide a predetermined pattern of cycling the expandable section 20 between the expanded state and the compressed state. In one embodiment of the present disclosure, the system 100 further comprises a pump unit 104 that is controlled by the controller 102 to deliver and withdraw volumes of gas or other fluids into and from the body 12 according to the predetermined pattern. In one embodiment of the present disclosure, the pump unit 104 may be a reciprocating piston pump and the controller 102 controls a displacement volume and frequency of the pump unit 104 to cause the expandable section 20 to cycle between the expanded state and the compressed state according to the predetermined pattern. As will be appreciated by one skilled in the art, a variety of other types of pumps may be used as the pump unit 104. A connection conduit 106 provides fluid communication between an output of the pump unit 104 and the central bore of the body 12 at the first end 14. Optionally, a further connection 108 may be provided between the first end 14 and the connection conduit 106.

One further embodiment of the system 100 may include a pressure monitor (not shown), such as a manometer or other type of pressure monitor, that monitors and generating an output signal of the expansion pressure in the expandable section 20. Information regarding the expansion pressure may allow the user to monitor pressure within the subject's esophagus 200 in order to reduce the risk of esophageal injury. The controller 102 may control the output of the pump unit 104 to deliver a predetermined volume of gas or other fluid into the expandable section 20. Alternatively, the controller 102 may expand the expandable section 20 to a set expansion pressure based upon the controller 102 receiving the pressure monitor's output signal. Alarm mechanisms can also be incorporated into the controller 102 and operatively coupled to the pressure monitor to generate an audio and/or visual alarm for altering a user to excessive expansion pressures or excessive expansion of the expandable section 20. Excessive expansion pressures and excessive expansion of the expandable section 20 are above a pre-determined threshold and can cause esophageal injury. The controller 102 may also cause the pump unit 104 to operate according to the predetermined pattern for example, by delivering a desired ratio of expansion to compression and to maintain the expandable section 20 in the expanded state for a specific period of time.

FIG. 3 also shows the sensor output 24 as connected to a display component 108 of the control box 102. In the embodiment of the TAC-B 10B that includes a sensor 22 that detects changes in pH, the display component 108 is a pH meter with an analogue or digital readout.

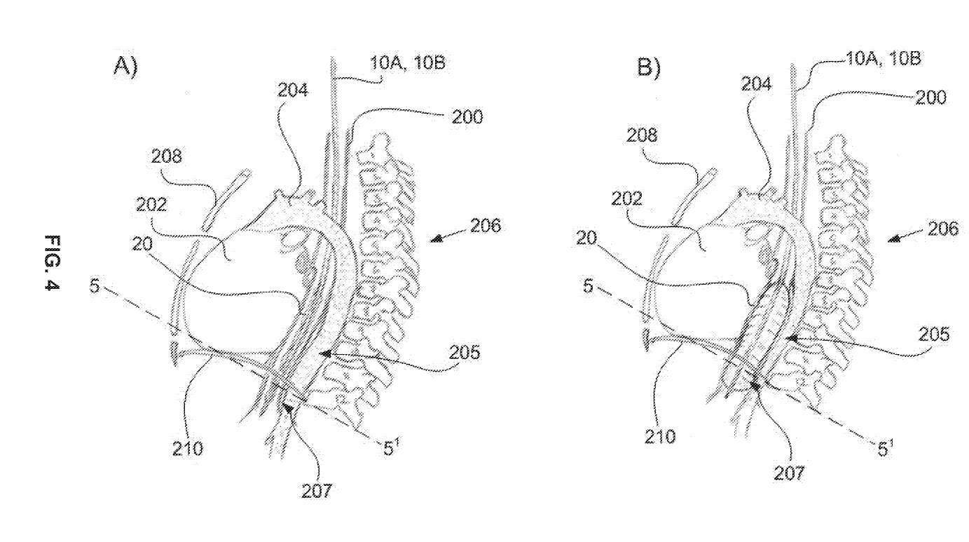

FIG. 4 shows the TAC-B 10A, B in a desired position within a deeply sedated or anesthetized subject. For perspective, FIG. 4 is a cross-sectional view through the subject's mid-section and it shows at least the following anatomical structures of the subject: esophagus 200, heart 202, aortic arch 204, descending aorta 205, spine 206, aortic hiatus 207, sternum 208 and diaphragm 210. The TAC-B 10A, B is inserted into the subject's esophagus 200 via the mouth. The TAC-B 10A, B is advanced down the esophagus 200 until the second end 16 lies just within the stomach. As described above, TAC-B 10B may provide a signal to the user that the second end 16 has entered the low pH environment of the stomach. When the TAC-B 10A, B is in the desired position, the expandable section 20 will be located within the esophagus 200 substantially adjacent and ventral to the descending aorta 205 extending from about the aortic hiatus 207 of the diaphragm 210 to about the aortic arch 204. This length of the expandable section 20 may be determined based upon the age and size of the subject. It is expected that the length of the expandable section 20 will vary from about 4.5 cm for pediatric and veterinary subjects up to about 22 cm in an adult human subject.

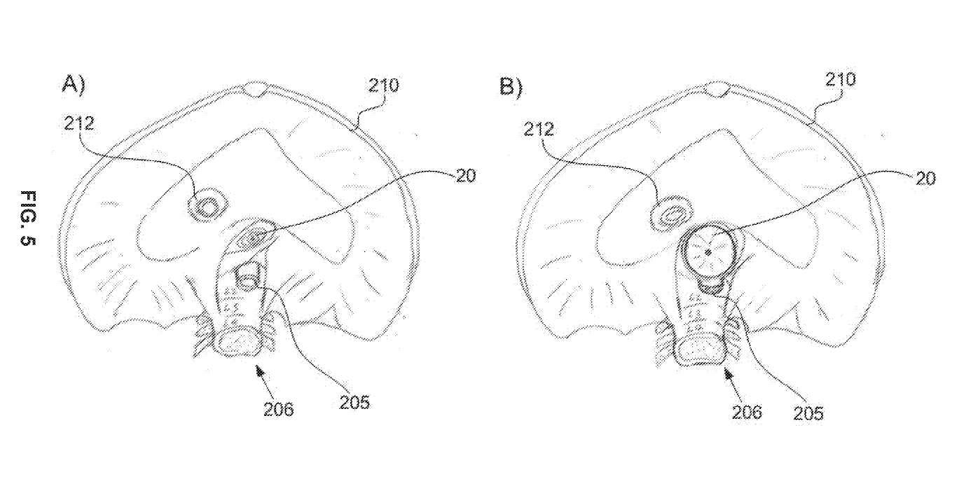

When the expandable section 20 is in the desired position and it is cycled to the expanded state, the expanded expandable section 20 applies pressure and causes at least partial compression of the descending aorta 205 at a point of compression. This compression of the descending aorta 205 reduces blood flow below the point of compression, such as within the abdomen and distal limbs. For clarity, below the point of compression is in reference to the direction of blood flow from the heart 202 through the descending aorta 205. As shown by contrasting FIG. 5A with FIG. 5B, when the expandable section 20 is in the expanded state it may also compress the vena cava 212, which can decrease blood flow retuning to the heart 202. The expanded TAC-B 10A, B will not compress the aortic root or ascending aorta of the heart 202 and, as such, cerebral and cardiac blood flow will remain substantially unaffected.

When the TAC-B 10A, B is expanded and held in the expanded position hemostasis may be achieved by the resultant reduction in blood flow to the abdomen and distal limbs.

Studies performed in swine resulted in the observation that if the expandable section 20 is maintained in the expanded state for about 60 seconds then the result will be about a 63% reduction in blood flow at the femoral artery. At the same time, cerebral blood flow is substantially preserved with 63% of normal carotid flow retained. If the expandable section 20 is held in the expanded state for longer than 60 seconds abdominal blood flow will continue to decrease but so too will carotid blood flow. Without being bound by any particular theory, it is postulated that the decrease in carotid blood flow may be due to pooling of venous blood in the abdomen and a drop in venous return to the heart 202.

In order to maximize reduction of flow to the abdomen, while retaining adequate flow to the brain for prolonged periods, the expandable section 202 may be cycled between the expanded state and the compressed state. Several ratios of expansion to compression have been explored. For example, a ratio of 8:2 of seconds of expansion to seconds of compression caused a 44% reduction of blood flow to the femoral artery while preserving 71% of resting carotid blood flow. Cycling the TAC-B 10A, B between the expanded state and the compressed state substantially decreases, but does not completely stop, abdominal or distal limb bleeding.

In one embodiment of the present disclosure, the TAC-B 10A, B may be made for single use and disposable and other components of the system 100 may be used repeatedly.

In another embodiment of the present disclosure, the system 100 may also be used as a platform for other means of monitoring or intervening in the subject's health.

I claim:

1. A system for controlling hemorrhage comprising:
(a) a transesophageal balloon that comprises a body and an expandable section that can cycle between an expanded state and a compressed state; and
(b) a controller for controlling actuating of the expandable section between the expanded state and the compressed state according to a predetermined pattern,
wherein the transesophageal balloon is positionable within a subject's esophagus adjacent the subject's aorta and when in the expanded state and positioned adjacent the subject's aorta, the transesophageal balloon is configured to at least partially compress a portion of the subject's aorta for decreasing hemorrhage below a point of compression.

2. The system of claim 1 further comprising a pump unit and a connection conduit that provides fluid communication between the pump unit and a central bore of the body, wherein a displacement volume and a frequency of the pump unit is controlled by the controller.

3. The system of claim 2 further comprising a pressure monitor for monitoring an expansion pressure of the expandable section, the pressure monitor also for generating an output signal that is received by the controller.

4. The system of claim 3 further comprising an alarm mechanism that is operatively coupled to the pressure monitor for alerting the user when the expansion pressure of the expandable section is above a predetermined threshold.

5. The system of claim 3 further comprising an alarm mechanism that is operatively coupled to the pressure monitor for alerting the user when the expansion of the expandable section is above a predetermined threshold.

6. The system of claim 1 further comprising a position sensor positioned at or near the expandable section, the position sensor for providing a positional signal of the transesophageal balloon.

7. The system of claim 6, wherein the sensor is a pH sensor.

8. An apparatus for controlling hemorrhage comprising:
(a) a tubular and elongate body with a first end, a second end and a central bore defined therebetween;
(b) one or more apertures that are defined at the second end and that provide fluid communication between the central bore and outside of the body;
(c) an expandable section that is attached to an outer surface of the body at the second end, wherein the expandable section is configured to move between an expanded state and a compressed state, when in the expanded state the expandable section has received and retained fluid through the one or more apertures and when in the compressed state the expandable section releases fluid through the one or more apertures,
wherein the apparatus is positionable within a subject's esophagus adjacent the subject's aorta.

9. The apparatus of claim 8, wherein the second end comprises a blunt tip.

10. The apparatus of claim 8, wherein the one or more apertures are positioned within a distance from the second end that is about ⅙ to about ⅓ of a distance between the first end and the second end.

11. The apparatus of claim 10, wherein the body has a length of between about 25 cm and about 125 cm.

12. The apparatus of claim 8, wherein the expandable section has a length of between about 4.5 cm and about 22 cm.

13. The apparatus of claim 8, wherein the body has an external diameter between about 0.25 cm and about 1.75 cm.

14. The apparatus of claim 8, further comprising a position sensor positioned at or near the expandable section, the position sensor for providing a positional signal of the transesophageal balloon.

15. The apparatus of claim 8, wherein the body further comprises line markings of 1 cm increments or 5 cm increments.

16. The apparatus of claim 8, wherein the first end is connectible with a connection conduit that provides fluid communication between the first end and a pump unit.

17. A method of controlling hemorrhage within a subject the method comprising steps of:
(a) inserting a transesophageal balloon within the subject's esophagus adjacent the subject's aorta;
(b) expanding the transesophageal balloon for establishing a point of compression of the subject's aorta and reducing blood flow beyond the point of compression; and
(c) contracting the transesophageal balloon for resuming blood flow beyond the point of compression.

18. The method of claim 17 further comprising a step (d) of holding the transesophageal balloon at a predetermined expansion pressure following step (b).

19. The method of claim 18 further comprising a step of cycling between the step (b) and the step (c).

20. The method of claim 19, wherein the step of cycling is performed at a ratio of 8 seconds of step (d) to 2 seconds of step (c).
